SEQUENCE LISTING (34 sequences)

SEQ ID NO: 1 - DNA, 7437 bp, synthetic construct
  source 1..7437: mol_type = other DNA
atgactgagaccatcctggccgcccaaatcgaggtcggcgagcaccacacggccacctggctcggtatgacggtcaacaccgacaccgtgttgtcgacggcgatcgccgggttgatcgtgatcgcgttggccttttacctgcgcgccaaagtgacttcgacggatgtgccaggcggggtgcagttgttttttgaggcgatcaccattcagatgcgcaatcaggtcgaaagcgccatcgggatgcggatcgcacccttcgtgctgccgctggcggtgaccatcttcgtgttcatcctgatctccaactggctggcagtcctcccggtgcagtacaccgataaacacgggcacaccaccgagttgctcaaatcggcagcagcggacatcaattacgtgctggcgctggcgcttttcgtgttcgtctgctaccacacggccggtatttggcggcgcggtattgtcggacacccgatcaagttgctgaaagggcacgtgacgctcctcgcgccgatcaaccttgtcgaagaagtcgccaagccaatctcgttgtcgctccgacttttcggcaacattttcgccggcggcattctggtcgcactgatcgcgctctttcccccctacatcatgtgggcgcccaatgcgatctggaaagcatttgacctgttcgtcggcgcaatccaggccttcatttttgcgctgctgacaattttgtacttcagccaagcgatggagctcgaagaggaacaccactagtaccggatgctggtaacggctaccagagccatcaaggaggataaggaaatggaccccactatcgctgccggcgccctcatcggcggtggactgatcatggccggtggcgccatcggcgccggtatcggtgacggtgtcgccggtaacgcgcttatctccggtgtcgcccggcaacccgaggcgcaagggcggctgttcacaccgttcttcatcaccgtcggtttggttgaggcggcatacttcatcaacctggcgtttatggcgctgttcgtcttcgctacacccgtcaagtaattcgacggcaaatggttgcaataggtagcaatgggtgaagtgagcgcgattgtcctggccgccagtcaggcggcagaggaaggcggcgagtccagcaacttcctcattcccaacggcacgtttttcgttgtgctggccatcttcctggtggtgctcgctgtcattggcactttcgtggtgccgccgatcttgaaggtcttgcgggaacgtgacgctatggtcgccaaaacgctggccgacaacaagaagtcggacgagcagttcgccgccgcacaggccgattacgacgaagccatgacggaagcccgagtccaggcgtcgtccttgcgcgacaatgcccgggcagatggccgtaaagtcatcgaggacgcacgcgtccgggccgaacaacaggtggcatcgacgttgcagaccgcccatgagcaattgaagcgggagagggacgccgtggaactcgatctgcgtgcccacgtgggcaccatgtcggcgactctggccagtcgaattctcggtgttgacctcaccgcttcagccgcgacgaggtaaccacgaatgtcgacgtttatcggacagctgttcgggttcgcggtcatcgtttatctggtgtggcgatttatcgtgccgctcgtagggcgtttgatgtccgcacggcaggacacggtgcgccaacagctggcggatgcggcggcggccgccgaccggctggcggaggcgagtcaagctcacacgaaggcgctggaagacgccaagtcggaagcgcaccgtgttgtggaagaggccaggacagatgccgaacgcatcgcagaacaactagaggcccaggccgacgtcgaggcggagcgcatcaaaatgcagggtgcccgtcaggtcgacctcatccgggcacagctgacccgtcagcttcgcctcgagctcggtcacgaatcggtgcgccaggcaagggaattggtacgcaatcacgtggccgatcaggcacaacaatcggccaccgtcgaccgcttcctggatcagctcgatgcgatggcgccggctacggccgatgtcgattacccactgctggccaagatgcgctcagccagccggagggcattaaccagcctggtggattggttcggcaccatggcccaggacctcgaccatcaaggtctgaccaccctcgccggcgagctggtgtcggtagcaagactgctggaccgcgaggccgtcgtcacccgctatctcaccgtgccagccgaagatgcgacgcccaggatccggctgatcgaacggctggtgtccggcaaggtcggcgcgccaacgctcgaggtgttgcgcacagccgtatcgaagcgctggtcggccaattccgatttgatcgatgcgatcgaacacgtgtcgcggcaggcgctgttagaactcgccgaacgtgcgggtcaggtcgacgaggtggaagaccagttattccggttttcccgcattctcgacgtgcagccccggcttgccatcctgttgggtgactgtgccgttccggccgaaggccgagtccggttgctgcgcaaggtgcttgagcgtgccgacagtaccgtcaacccggtcgtggtcgcgctgttgtctcacaccgtcgagctgctgcggggtcaggcagttgaggaagcggtgctgttcctggccgaagttgcggtggctcgccgcggcgaaatcgtcgcgcaggtcggcgcggcggccgagctcagcgatgctcagcgcactcgcctcaccgaagtgctgagccgtatctacggtcaccccgtgaccgtgcagctgcatatcgacgccgcgctgctgggcggattgtccatcgcggtcggtgacgaagtgatcgacggtacgctctcgtctcgtctagctgcggccgaggcacgactgcccgactgaacccgaactagtcagcacaaaccgaagtaggaagacgaaaagctatggctgagttgacaatccccgctgatgacatccagagcgcaatcgaagagtacgtaagctctttcaccgccgacaccagtagagaggaagtcggtaccgtcgtcgatgccggggacggcatcgcacacgtcgagggtttgccatcggtgatgacccaagagctgctcgaattcccgggcggaatcctcggcgtcgccctcaacctcgacgagcacagcgtcggcgcggtgatcctcggtgacttcgagaacatcgaagaaggtcagcaggtcaagcgcaccggcgaagtcttatcggttccggttggcgacgggtttttggggcgggtggttaacccgctcggccagccgatcgacgggcgcggagacgtcgactccgatactcggcgcgcgctggagctccaggcgccctcggtggtgcaccggcaaggcgtgaaggagccgttgcagaccgggatcaaggcgattgacgcgatgaccccgatcggccgcggccagcgccagctgatcatcggcgaccgcaagaccggcaaaaccgccgtctgcgtcgacaccatcctcaaccagcggcagaactgggagtccggtgatcccaagaagcaggtgcgctgtgtatacgtggccatcgggcagaagggaactaccatcgccgcggtacgccgcacactggaagagggcggtgcgatggactacaccaccatcgtcgcggccgcggcgtcggagtccgccggtttcaaatggcttgcgccgtacaccggttcggcgatcgcccagcactggatgtacgagggcaagcatgtgctgatcatcttcgacgacctgactaagcaggccgaggcataccgggcgatctcgctgctgctgcgccgtccgcccggccgtgaggcctaccccggcgatgtgttctatctgcattcgcggcttttggagcgctgcgccaaactgtccgacgatctcggtggcggctcgctaacgggtctgccgatcatcgagaccaaggccaacgacatctcggcctacatcccgaccaacgtcatctcgatcaccgacgggcaatgtttcctggaaaccgacctgttcaaccagggcgtccggccggccatcaacgtcggtgtgtcggtgtcccgagtcggcggcgcggcgcagatcaaggctatgaaagaggtcgccggaagcctccgcttggacctttcgcaataccgcgagctagaagctttcgccgctttcgcttctgatttggacgccgcatcgaaggcgcagttggagcgcggcgcccggctggtcgagctgctcaagcagccgcaatcccagcccatgcccgttgaggagcaagtggtttcgatcttcctgggcaccggcggtcacctggactcggtgcccgtcgaggacgtccggcggttcgaaaccgaattactggaccacatgcgggcctccgaagaagagattttgactgagatccgggacagccaaaagctcaccgaggaggccgccgacaagctcaccgaggtcatcaagaacttcaagaagggcttcgcggccaccggtggcggctctgtggtgcccgacgaacatgtcgaggccctcgacgaggataagctcgccaaggaagccgtgaaggtcaaaaagccggcgccgaagaagaagaaatagctaaccatggctgccacacttcgcgaactacgcgggcggatccgctcggcagggtcgatcaaaaagatcaccaaggcccaggagctgattgcgacatcgcgcatcgccagggcgcaggctcggctcgagtccgctcggccctacgcttttgagatcacccggatgcttaccaccctggccgctgaagccgcactggaccatccgttgctcgtcgagcgcccggagccgaaacgagccggcgtgctggtggtgtcgtccgatcgtggtttgtgcggcgcatacaacgccaatattttccgtcgctccgaggagctgttctccctgctgagggaggccggaaagcagccggtgctgtatgtggtgggccgtaaggcgcagaactactacagttttcggaactggaacatcaccgagtcgtggatgggtttctccgagcaacccacgtacgagaacgccgccgagatcgcttcgaccttagtggatgcgttcctgctcggcaccgacaacggcgaggatcaacggtccgacagcggcgagggcgtcgacgaactgcacatcgtttacaccgagttcaagtcgatgctgtcgcaatcggcggaggctcaccggatcgcccccatggtggtggagtacgtcgaggaagacatcggaccgcgcacgctgtactcgttcgagcccgacgcgacgatgctgttcgagtcattgttgccgcgctacctgactacccgggtgtacgcggcgctgctggagtccgcggcgtcggagcttgcctcgcggcaacgtgcgatgaagtcggccaccgacaacgccgatgacctcatcaaggccctgacgctgatggcaaaccgcgagcggcaggcccagatcacccaggagattagtgaaatcgtcggtggcgcaaatgcgctcgccgaagcccgctaggcccaagctaggttagccccacgaggaagcgaagaagatatgactaccactgccgaaaagaccgaccggccgggaaagccgggaagctccgacaccagcggccgcgtggtacgggtcactgggcccgtcgtcgacgtcgagtttcctcgcggttccatccccgagctgttcaatgcactgcacgctgagatcaccttcgagtcgctggcgaaaaccctcaccttggaggtggcgcagcacctcggcgacaacctggtgcgcaccatctcgctgcagccgaccgacggcttggtgcgcggcgtcgaggtgatcgacaccgggaggtcgatctcggtgccggtcggtgagggtgtgaagggccacgtcttcaatgcgctgggagattgcctggacgagccgggatatggcgaaaaattcgaacactggtcgattcaccgcaagccgccggcgttcgaggagctggagcctcggaccgagatgctcgagaccggtctgaaggtggtcgacctgctgactccgtatgttcgtggcggcaagatcgcactgttcggcggtgccggggtgggcaagacggtgctgattcaggagatgatcaaccgcatcgcccgtaacttcggtggtacgtcggtgttcgccggagtgggcgagcgcacccgcgagggcaacgatctgtgggtcgagcttgccgaagccaacgtgctcaaggacaccgcgctggtattcggacagatggacgagccgccgggcacccgtatgcgtgttgcgctgtctgcgctgacgatggcggagtggttccgtgacgagcagggtcaagacgtattgctgttcatcgacaacatcttccggttcacccaggctgggtcggaagtgtcgacgcttctcggccggatgccgtcggccgtgggataccagcccacgctggccgacgagatgggcgagctgcaggagcgcatcacctcgacgcggggacgctcgatcacgtcgatgcaagccgtctacgtgcccgccgacgactacaccgacccagcgccggcgaccacgttcgcccacctggacgccacgaccgagctatcccgtgcggtgttctccaagggcatcttccccgccgtggacccgctggcgtccagctcgaccatcctggaccccagcgttgtcggggatgagcactaccgcgtggcccaggaagtcatccggatcctgcagcgttacaaggaccttcaggacattatcgcgatcctcggtatcgacgagttgtcggaggaggacaagcagctggtgaaccgcgcccggcgtatcgagcggttcctatcgcagaacatgatggcagccgaacagttcaccggccagccgggttcgaccgtcccggtgaaggagaccattgaagcgttcgaccgcttgtgcaagggcgatttcgatcacgtacccgaacaggccttcttcttgatcggtggccttgatgacctggccaagaaagccgagagtctcggcgccaagctgtgacgggagttgtggcatggccgaattgaacgttgagatcgtcgccgtcgaccggaacatctggtcgggtacggcgaagtttctgttcacccgcaccaccgtcggtgagatcggcatcctgccccgccacattccgttggtggcccaattggtcgatgacgccatggtgcgggtcgagcgggagggagaaaaggacctgaggatcgcggtcgacggcgggttcctgtcggtgaccgaggagggcgtcagcattctcgccgaatctgccgagttcgagtcggagatcgacgaggccgccgccaagcaggattccgaatccgacgatccccgcatcgctgccaggggccgcgccagattgcgcgccgtcggcgcgatcgactaa

SEQ ID NO: 2 - DNA, 7472 bp, synthetic construct
  source 1..7472: mol_type = other DNA
gtgctggccgctgaagagggtggcgcagccatccacgtcggccatcacacgctcgtgttcgagctgttcggcatgacgttcaatggcgacaccattctcgccactgccgtcaccgccgtgatcgtgatcgcgctggccttctacctgcgggccaaggtcacctccaccggggtgcccagcggtgtgcagctgttctgggaggcgctgacgatccagatgcgtcagcagatcgagggctcgatcggcatgaagatcgccccgttcgtgctgccgctgtcggtgacgatcttcgtgttcatcctgatctccaactggctcgcggtgctcccgctgcagtacggcggagccgacggcgccgcggccgagttgtacaaggcacccgcctccgacatcaacttcgtgctggcgctcgcgctgttcgtgttcgtctgctaccacgcggcaggcatctggcgtcgcggcatcgtcggccacccgatcaaggttgtcaagggccacgtcgcgttcctcgcgccgatcaacatcgtcgaagaactcgccaagccgatctcgctggccctccgtcttttcggcaacatcttcgccggcggcatcctggtcgcgctgatcgccatgttcccctggtacatccagtggttccccaacgccgtgtggaagaccttcgacctgttcgtcggcctcatccaggccttcatcttctcgctgctgacgatcctgtacttcagccagtcgatggaactggaccacgaggaccactgacgagcaaccctgctggaccgaacaaatccctacgacccgatcgacacgaactctgacggcaacatttactgagccgccagttaccaaggaggaataaaggaatggatctcgatcccaacgccatcatcacggccggcgccctgatcggcggtggattgatcatgggtggcggcgccatcggtgccggtatcggcgacggtatcgcgggtaacgcgctgatctcgggtatcgcccgtcagcccgaggcccagggccggctgttcaccccgttcttcatcaccgtcggtctggtggaagccgcgtacttcatcaacctggccttcatggcgctgttcgtcttcgccactcctggccttcagtaatccgccggcatgggtgaattcagcgcaacgatcctggcggccagccaggcagccgaggaaggcgggggcggaagcaacttcctgatccccaacggcaccttcttcgccgtgctgatcattttcttgatcgtgctcggcgtgatctcgaaatgggttgtgccaccgatcagcaaggtgctggccgagcgggaagcgatgctggccaagaccgcagccgacaaccgcaagtccgccgagcaggtcgctgcggcacaggcggactacgagaaggaaatggccgaggcgcgtgcccaggcctcggcgcttcgcgacgaggcccgcgcggccggtcgctcggtggtcgacgagaagcgtgcacaagccagtggtgaggtggcccagaccctgacccaggcggaccagcagttgtccgcccagggtgatcaggtgcgcagtggcctcgagtcgtcggtggacgggctgtcagccaagctggccagcaggatcctcggcgtcgacgtgaattcaggtgggacacagtagatgtcgattttcatcggacagctgatcggctttgccgtcatcgcgttcatcatcgtcaagtgggtggtgccacccgtacggaccctgatgcgtaaccagcaggaggccgtgcgtgcggcgctcgccgagagtgccgaggcagccaagaagctcgctgatgccgacgcgatgcacgccaaggcgcttgccgacgccaaggccgagtcggagaaggtcaccgaggaggccaagcaggactccgagcgcatcgccgcgcagctgtccgaacaggccggctccgaggcggagcggatcaaggcccaaggcgcacagcagatccagttgatgcgccagcagctcatccgtcagctgcgcaccgggctcggcgcggaggccgtgaacaaggccgccgagatcgtccgggcgcacgtcgccgatccgcaggcacagtcggccaccgtcgaccgcttcctgagcgaactcgagcagatggcgccgtcgagcgtcgtcatcgacaccgccgcgacgagccggctgcgtgccgcgagccgccaatcgctcgcggcgctggtcgagaagttcgactcggtcgcgggcggcctcgacgccgacgggctcaccaacctcgccgacgaactggcctcggtcgcgaagctgctgctgagcgagaccgcgctcaacaagcacctggccgagcccaccgacgacagcgctcccaaggtgcgcctgctcgagcgtctgctgtcggacaaggtcagcgccaccacgctggacctgctgcgcaccgcggtgtcgaaccgctggtcgaccgagtcgaacctgatcgacgccgtcgagcacaccgcacgcctggccctgctcaagcgcgccgagatcgccggtgaggtcgacgaggtcgaggagcagctgttccgcttcggccgggtcctcgacgccgagccgcgcctgtcggcactgctgagcgactacaccacccccgcggaaggtcgtgtcgcgctgctggacaaggcgctcaccggtcgccccggcgtgaaccagacggcagcggccctgctgtcgcagaccgtcggactgctccgcggcgagcgcgccgacgaggccgtcatcgatctcgccgaactcgcggtgtcccggcgaggcgaggttgtggcgcatgtgtcggccgcggccgaactcagcgacgcacagcgcacgcgtctgaccgaggtgctctcccgcatctacggccgtccggtgtccgtgcaactccacgtcgacccggaactcctcggtggcctgtcgatcacggtcggtgacgaagtgatcgacggttccatcgcctcccgtttggccgccgcgcagaccggcttgccggactgacttgatccacgaaccacccaagaactaggtaggaagacgaaaaaccatggcagagttgacaatctcggctgctgatatcgaaggtgccatcgaggactacgtatcctcgttttccgccgacaccgagcgtgaagagatcggcaccgtcatcgacgccggtgacggcatcgcccacgtcgagggtctgccctcggtcatgacgcaggaactgctcgagttccccggcggggtcctcggcgtggcactgaacctcgacgagcacagcgtcggcgccgtcatcctgggtgagttcgagaagatcgaagagggccagcaggtcaagcggaccggcgaggtgctctcggtcccggtcggcgacgccttcctgggccgcgtcgtcaacccgctgggccagccgatcgacggccagggcgacatcgccgccgagacccgccgcgccctcgagctgcaggcgccttctgtggtgcagcgccagagcgtgtccgagccgctgcagaccggcatcaaggccatcgacgccatgaccccgatcggccgcggtcagcgccagctgatcatcggcgaccgcaagaccggcaagaccgccgtctgcgtcgacaccatcctcaaccagcgtgaggcctggctgaccggcgaccccaagcagcaggtccgctgcgtctacgtcgccatcggccagaagggcaccaccatcgccagcgtgaagcgcgcgctggaagagggcggcgccatggagtacacgacgatcgtcgccgccccggcatccgacgccgcaggcttcaagtggctggccccctacaccggctcggccatcggccagcactggatgtacaacggcaagcacgtcctgatcgtgttcgacgacctgtccaagcaggccgacgcctaccgcgcgatctcgctgctgctgcgccgcccgccgggccgcgaggcgttccccggtgacgtgttctacctgcactcgcgcctgctggagcgctgcgcgaagctgtcggacgaactcggcggcggttcgatgacgggtctgccgatcatcgagaccaaggccaacgacatctcggcgttcatcccgaccaacgtcatctcgatcaccgacggccagtgcttcctggagtccgacctgttcaaccagggtgtgcggccggccatcaacgtcggtgtgtcggtgtcccgcgtcggtggcgccgcccagatcaaggccatgaaagaggtcgcaggctcgctgcgtctggacctgtcgcagtaccgcgagctggaggccttcgcggccttcgcctcggacctcgacgccgcgtcgaaggcccagctggaccgcggtgcccgcctggtcgagctgctcaagcagccgcagtacagcccgctggccgtcgaggagcaggtcgtcgcgatcttcctcggcacccagggccacctggattccgttccggtcgaagacgttcagcgcttcgagtccgagctgctggagcacgtgaaggccagccactccgacatcttcgacggcatccgtgagaccaagaagctctccgaggaagccgaggagaagctggtctcggtcatcaacgaattcaagaagggcttccaggcctctgacggcagctcggtggtcgtctccgagaacgccgaggccctcgatcccgaggacctggagaaggaatccgtcaaggtccgcaagccggcacccaagaaggcctaggtaacagatggcagccacactgcgcgaactacgcgggcgcatccgctccgccgggtcgatcaagaagatcaccaaggcccaggagctgatcgcgacctcgcggatcgccaaggcgcaggcacgggtcgaagcagcccgtccctatgccgccgagatcaccaacatgctcaccgaacttgcgggtgccagtgcgctggaccacccgctgctcgtggagcgcaagcagcccaagcgggccggcgtgctggtggtgtcgtcggaccgcggcctgtgcggtgcctacaacgccaacgtgctgcgccgcgccgaggagctgttctcgctgctgcgcgacgagggcaaggacccggtgctgtacgtggtcggccgtaaggcgttgggctacttcagcttccggcagcgcaccgtggtcgagtcgtggaccggcttctccgagcgcccgacctacgagaacgccagggagatcgccgacaccttggtgaacgccttcatggcgggcgccgacgacgagggtgacgacgcgggcgccgacggcatcctcggggtcgacgaactgcacatcgtgttcaccgagttccggtcgatgctctcgcagaccgcggtcgcccgtcgtgccgcgccgatggaggtcgagtacgtcggcgaggtcgagaccgggccgcgcacgctctactcgttcgaaccggatccggagacgctgttcgacgcgttgttgccgcgctacatcgcgacccgcgtgtacgccgcacttctcgaggccgcagcgtccgagtcggcctcgcgccggcgcgccatgaagtcggccaccgacaacgccgacgacctcatcaaggcgctgacgctggcggcgaaccgcgagcgccaggcacagatcacccaggaaatcagcgagatcgtcggcggcgccaacgcgctggccggctcgaaataggctgagaagcaagcccctttcaggaagcgaagagagaatgactgctactgcagaaaagaccgcgggtcgcgtagtccgcatcaccggcccggtggtggacgtcgaattcccgcgtggctctgtgcccgagctgttcaacgcgctgcacgccgagatcacgttcggtgcgctcgccaagaccctgaccctcgaggtcgcccagcacctcggtgacagcctggtgcgctgcatctccatgcagcccaccgacggcctggtgcgtggcgtcgaggtcaccgacaccggcgcctcgatctccgtcccggtcggcgacggcgtcaagggccacgtgttcaacgcgctcggcgactgcctcgacgatccgggctacggcaaggacttcgagcactggtcgatccaccgcaagccgccggccttctctgacctggagccccgcaccgagatgctggagaccggtctgaaggtcgtcgacctgctcaccccgtacgtgcgtggcggcaagatcgccctgttcggcggcgcgggcgtgggcaagacggttctgatccaggagatgatcaaccgtatcgcccgcaacttcggtggtacctcggtgttcgccggcgtgggtgagcgcacccgtgagggcaacgacctgtgggtcgagctcgccgacgccaacgtgctcaaggacaccgcgttggtgttcggtcagatggacgagccgccgggcacccgtatgcgcgtcgccctgtcggccctgaccatggccgagttcttccgcgacgagcagggccaggacgtgctgctgttcatcgacaacatcttccggttcacccaggccggttccgaggtgtcgaccctgctgggtcgtatgccttcggccgtgggttaccagccgacgctggccgacgagatgggtgagctgcaggagcgcatcacctcgacccgtggtcgctccatcacctcgatgcaggccgtgtacgtgcccgccgacgactacaccgacccggccccggcgacgacgttcgcccacctggacgccaccaccgagctctcgcgtgcggtgttctcgaagggcatcttcccggcggtggatccgctggcgtcgtcctcgaccatcctggacccggcgatcgtcggtgacgagcactaccgcgtcgcccaggaagtcatccggatcctgcagcgctacaaggatctccaggacatcatcgcgatcctcggtatcgacgagctgtccgaagaggacaagcagctggtgaaccgggcgcgtcgtatcgagcgcttcctgagccagaacatgatggccgccgagcagttcaccggtcagccgggctcgaccgtgccgctcaaggagaccatcgaggcgttcgacaagctcaccaagggcgagttcgatcacctgcccgagcaggcgttcttcctgatcggtggtctggacgacctggcgaagaaggccgagagcctcggcgccaagctgtgattgccatgtccttgagcgtccgaaaggtggtgtggtgtggctgatctgaacgtcgagatcgtcgccgtcgagcgtgagctctggtccggacccgctacgttcgtgttcacccgcaccaccgccggtgagatcggcatcctgccgcggcacatcccgctcgtggcgcagctggtcgacgacgcgatggttcgggtcgagcgcgagggcgaggacgatctgcggatcgcggtcgacggcggtttcctgtcggtgaccgaggagaccgtccggatcctcgtggagaacgcacaattcgagtccgagatcgacgcggatgctgccaaggaagacgcagcatccgacgacgagcggaccgcggcatggggccgggcgcggctgcgcgccctcggccagatcgactag

SEQ ID NO: 3 - DNA, 9338 bp, synthetic construct
  source 1..9338: mol_type = other DNA
tgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatttactcatttattcatttgctcatttactctggtagctgcgcgatgtattcagatagcagctcggtaatggtcttgccttggcgtaccgcgtacatcttcagcttggtgtnatcntccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtccagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttaaagtactttaaagtactttaaagtactttgatcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatccaagctcaagctcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtcgagtggagatgtggagtgggcgcttacacagtacacgaggacttctagagaattctagaggtccgctttgaatcgaactaatattttttttggtgaatcgcattctgactggttgcctgtcagaggcggagaatccggtgattttgtttttcgacgtggtgacgggcatgccttcgcgaaaatcgcacctgcttcccgccgcggtgagctcgctggagagcgtgaccgcctcatttggctcaaaggtcgaggtgtggcttgccccgaggtgatcaactggcaggaggaacaggagggtgcatgcttggtgataacggcaattccgggagtaccggcggctgatctgtctggagcggatttgctcaaagcgtggccgtcaatggggcagcaacttggcgctgttcacagcctatcggttgatcaatgtccgtttgagcgcaggctgtcgcgaatgttcggacgcgccgttgatgtggtgtcccgcaatgccgtcaatcccgacttcttaccggacgaggacaagagtacgccgcagctcgatcttttggctcgtgtcgaacgagagctaccggtgcggctcgaccaagagcgcaccgatatggttgtttgccatggtgatccctgcatgccgaacttcatggtggaccctaaaactcttcaatgcacgggtctgatcgaccttgggcggctcggaacagcagatcgctatgccgatttggcactcatgattgctaacgccgaagagaactgggcagcgccagatgaagcagagcgcgccttcgctgtcctattcaatgtattggggatcgaagcccccgaccgcgaacgccttgccttctatctgcgattggaccctctgacttggggttgaactagtgatcgatatcgatatcctcaattccgtcggtcgattacaatcacatgacttggcttccatttcactactattatgcacacccactacatacatgatcatataaccaattgccctcatccccatcctttaactatagcgaaatggattgattgtttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgttgacggttgtgtatggaagattgagtgaagtacggttgaactttctgaagagaggttagaaaaagaggtagagagaaaagaaagagcagatccaagaaatagcaaagtggagcccgctcgaggaggtcaaagcttaccagatacgattgcttgacgaaccgtggtatgaagacggtgatgagaagagggtgcgggaggggtggcctttttgagtgtgagtgagtgggcgcactgactaagcgagccaaagggtctagcttctgttgccccgccaagctagtttttttctctcacttttttgctgcttcgttgggagcccaccgagcggagccagtagaaaaaatgatgtaacggatgatgagccaactgtcgtttctttgcttgctatgcactgcggtggcggaggcaaaccgctcgaatcgagaatctatgtgtgtatctttcacttgcggtgtctcaacctggccacagctatcgcattccacgtgataatatgctcaatgaattgctcttcgatgctttctaccgaatctagtcgtgccgatatgcctattattccctctgtatagccgctcactgcccatgcttggtgtactatcgataacgtttgggcaccactgagggacagccgccgctttgcgctcctccaatttttttcccggcacaccgctcgtcctgtcacatcttaccgttgcttgcagctcaccatttgccgcagtggatagtcgcgcgtctagcctggcctgttgctaatttgtatctgaaccatcctttgaccaccgtttgctggatccgcggccgcgaattggaattaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatccagatcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgatcctgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaaacgcctggtatctttatagtcctgtcgggtttcgccacctctgactggagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggct

SEQ ID NO: 4 - DNA, 1032 bp, synthetic construct
  source 1..1032: mol_type = other DNA
gaattctagaggtccgctgtgacacaagaatccctttacttctcgaccgtattgattcggatgattcctacgcgagcctgcggaacgaccaggaattctgggagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggtgccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggccggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgaactagtgatcgatatc

SEQ ID NO: 5 - DNA, 25 bp, synthetic construct
  source 1..25: mol_type = other DNA
gatatcctcaattccgtcggtcgat

SEQ ID NO: 6 - DNA, 27 bp, synthetic construct
  source 1..27: mol_type = other DNA
tctagaagtcctcgtgtactgtgtaag

SEQ ID NO: 7 - DNA, 40 bp, synthetic construct
  source 1..40: mol_type = other DNA
agtacacgaggacttctagaagccgttgcgtttgacggcc

SEQ ID NO: 8 - DNA, 42 bp, synthetic construct
  source 1..42: mol_type = other DNA
gccaggatggtctcagtcatggcccaaagcctagccggtgac

SEQ ID NO: 9 - DNA, 41 bp, synthetic construct
  source 1..41: mol_type = other DNA
atcgatatccagccggatcgacttcgccgggtcaccccggc

SEQ ID NO: 10 - DNA, 40 bp, synthetic construct
  source 1..40: mol_type = other DNA
ccgacggaattgaggatatcccagggtgacccacagcaac

SEQ ID NO: 11 - DNA, 50 bp, synthetic construct
  source 1..50: mol_type = other DNA
gcgcgccgtcggcgcgatcgactaagaattctagaggtccgctttgaatc

SEQ ID NO: 12 - DNA, 40 bp, synthetic construct
  source 1..40: mol_type = other DNA
cggcgaagtcgatccggctggatatcgatcactagttcaa

SEQ ID NO: 13 - DNA, 42 bp, synthetic construct
  source 1..42: mol_type = other DNA
gtcaccggctaggctttgggccatgactgagaccatcctggc

SEQ ID NO: 14 - DNA, 50 bp, synthetic construct
  source 1..50: mol_type = other DNA
aaagcggacctctagaattcttagtcgatcgcgccgacggcgcgcaatct

SEQ ID NO: 15 - DNA, 35 bp, synthetic construct
  source 1..35: mol_type = other DNA
aaccgtctgcgtgaggcggagtgggacgtgaccga

SEQ ID NO: 16 - DNA, 35 bp, synthetic construct
  source 1..35: mol_type = other DNA
cggccgaccacttgccgaacggatgggttcccgca

SEQ ID NO: 17 - DNA, 45 bp, synthetic construct
  source 1..45: mol_type = other DNA
gatatcctcaattccgtcggtcgattacaatcacatgacttggct

SEQ ID NO: 18 - DNA, 41 bp, synthetic construct
  source 1..41: mol_type = other DNA
tctagaagtcctcgtgtactgtgtaagcgcccactccacat

SEQ ID NO: 19 - DNA, 31 bp, synthetic construct
  source 1..31: mol_type = other DNA
gaattctagaggtccgctgtgacacaagaat

SEQ ID NO: 20 - DNA, 35 bp, synthetic construct
  source 1..35: mol_type = other DNA
gatatcgatcactagttcaggcgccgggggcggtg

SEQ ID NO: 21 - DNA, 40 bp, synthetic construct
  source 1..40: mol_type = other DNA
cacagtacacgaggacttctagaggtggcggcggtcttga

SEQ ID NO: 22 - DNA, 47 bp, synthetic construct
  source 1..47: mol_type = other DNA
gtcacagcggacctctagaattcggtcgttgtctgagtcatggaggt

SEQ ID NO: 23 - DNA, 46 bp, synthetic construct
  source 1..46: mol_type = other DNA
actagtgatcgatatctcacatcgatgagcgcgtccatgatcggca

SEQ ID NO: 24 - DNA, 40 bp, synthetic construct
  source 1..40: mol_type = other DNA
gacggaattgaggatatcgtgcatgtccagcggccgcgac

SEQ ID NO: 25 - DNA, 45 bp, synthetic construct; identical to SEQ ID NO: 17
  source 1..45: mol_type = other DNA

SEQ ID NO: 26 - DNA, 41 bp, synthetic construct; identical to SEQ ID NO: 18
  source 1..41: mol_type = other DNA

SEQ ID NO: 27 - DNA, 31 bp, synthetic construct; identical to SEQ ID NO: 19
  source 1..31: mol_type = other DNA

SEQ ID NO: 28 - DNA, 35 bp, synthetic construct; identical to SEQ ID NO: 20
  source 1..35: mol_type = other DNA

SEQ ID NO: 29 - DNA, 39 bp, synthetic construct
  source 1..39: mol_type = other DNA
tgcgttgatacatcgcccaggtggcctcgatctcggcgt

SEQ ID NO: 30 - DNA, 38 bp, synthetic construct
  source 1..38: mol_type = other DNA
tgctgcgggtcgacgccggtgaccgtgatgtcacccct

SEQ ID NO: 31 - DNA, 48 bp, synthetic construct
  source 1..48: mol_type = other DNA
agttggatcctacttccaatccaatgctatgactgagaccatcctggc

SEQ ID NO: 32 - DNA, 46 bp, synthetic construct
  source 1..46: mol_type = other DNA
cttttatcccacccaaatgttagtcgatcgcgccgacggcgcgcaa

SEQ ID NO: 33 - DNA, 16 bp, synthetic construct
  source 1..16: mol_type = other DNA
catttgggtgggataa

SEQ ID NO: 34 - DNA, 18 bp, synthetic construct
  source 1..18: mol_type = other DNA
agcattggattggaagta

(19) United States
(12) Patent Application Publication
(10) Pub. No.: US 2024/0360459 A1
(43) Pub. Date: Oct. 31, 2024

(54) MYCOBACTERIUM TUBERCULOSIS (Mtb) ADENOSINE TRIPHOSPHATE (ATP) SYNTHASE-EXPRESSING RECOMBINANT BACTERIUM, AND CONSTRUCTION METHOD AND EXPRESSION METHOD THEREOF

(71) Applicant: Nankai University, Tianjin (CN)

(72) Inventors: Hongri Gong, Tianjin (CN); Yuying Zhang, Tianjin (CN); Zihe Rao, Tianjin (CN)

(21) Appl. No.: 18/437,991

(22) Filed: Feb. 9, 2024

(30) Foreign Application Priority Data
Apr. 27, 2023 (CN) 202310468268.7

(51) Int. Cl. C12N 15/70; C07K 14/35; C12N 1/20; C12N 9/00; C12R 1/19

(52) U.S. Cl. CPC C12N 15/70; C07K 14/35; C12N 1/20; C12N 9/00; C12R 2001/19

(57) ABSTRACT

The present disclosure provides a Mycobacterium tuberculosis (Mtb) adenosine triphosphate (ATP) synthase-expressing recombinant bacterium, and a construction method and an expression method thereof, and belongs to the technical field of exogenous expression. The present disclosure provides a construction method of an Mtb ATP synthase-expressing recombinant bacterium, including the following steps: using a Mycobacterium smegmatis (Msm) competent cell containing an auxiliary gene knockout plasmid as a basal cell; transferring an Mtb ATP synthase gene cluster with an affinity purification tag into the basal cell, knocking out a Msm ATP synthase genome, and collecting a strain without the auxiliary gene knockout plasmid after conducting repeated subculture; and transferring the Mtb ATP synthase gene cluster into the strain by prokaryotic expression to obtain the Mtb ATP synthase-expressing recombinant bacterium.

### CROSS REFERENCE TO RELATED APPLICATION

This patent application claims the benefit and priority of Chinese Patent Application No. 202310468268.7, filed with the China National Intellectual Property Administration on Apr. 27, 2023, the disclosure of which is incorporated by reference herein in its entirety as part of the present application.

### REFERENCE TO SEQUENCE LISTING

A computer readable XML file entitled “SEQUENCE LISTING”, that was created on Jan. 11, 2024, with a file size of about 56598 bytes, contains the sequence listing for this application, has been filed with this application, and is hereby incorporated by reference in its entirety.

### TECHNICAL FIELD

The present disclosure belongs to the technical field of exogenous expression, and specifically relates to a Mycobacterium tuberculosis (Mtb) adenosine triphosphate (ATP) synthase-expressing recombinant bacterium, and a construction method and an expression method thereof.

### BACKGROUND

Tuberculosis (TB), an infectious disease caused by Mtb, has the highest mortality rate caused by a single causative agent for many years before the COVID-19 outbreak. “Global Tuberculosis (TB) Report 2021” released by the World Health Organization (WHO) states that there are approximately 2 billion people latently infected with TB in the world. The TB caused by multi-drug-resistant and extensively-drug-resistant Mtb strains is a serious threat to the health of all mankind. To achieve control of TB, new anti-TB drugs are urgently needed. Since Mtb and related mycobacteria cannot obtain sufficient energy through substrate-level phosphorylation pathways, their growth requires an oxidative phosphorylation system to maintain energy supply. In view of this, an Mtb oxidative phosphorylation system has become an important target for the development of novel anti-drug-sensitive and drug-resistant TB. Membrane protein complexes NDH-1, NDH-2, SDH-1, SDH-2, Cytbcc/aa₃, and Cytbd in the oxidative phosphorylation system are accompanied by a transmembrane proton transport force in the process of transferring electrons. The proton transport force is then used to produce directly usable high-energy molecule ATPs by ATP synthases. ATP synthesis plays an important role in Mtb growth and survival. However, the composition and functional mechanism of Mtb ATP synthase, as well as the mechanism of action in related small-molecule inhibitors are still unclear, restricting the development of novel inhibitors with a higher activity.

Due to the difficulty in expression and purification, the structure and function of Mtb ATP synthase and the mechanism of action of associated inhibitors are currently deduced by studying Mycobacterium smegmatis (Msm) ATP synthase. However, based on genome sequence analysis, it is found that compared to the Msm ATP synthase, the Mtb ATP synthase shows differences in drug targets. Accordingly, the precise assembly and functional mechanisms of Mtb ATP synthase still need to be improved to better develop ideal anti-TB drugs for the drug targets.

### SUMMARY

An objective of the present disclosure is to provide a Mtb ATP synthase-expressing recombinant bacterium, and a construction method and an expression method thereof. The present disclosure solves the problem of difficulty in knocking out background genes of essential genes for heterologous expression, and successfully expresses and purifies the Mtb ATP synthase in Msm exogenously. The present disclosure avoids the biological hazards of Mtb, and has simple operation, low cost, and high safety.

The present disclosure provides a construction method of a Mtb ATP synthase-expressing recombinant bacterium, including the following steps: (1) transferring an Mtb ATP synthase gene cluster with an affinity purification tag into a Msm competent cell to obtain a strain a; where the Msm competent cell carries an auxiliary gene knockout plasmid;
(2) knocking out a Msm ATP synthase genome in the strain a to obtain a strain b;
(3) subjecting the strain b to repeated subculture on a kanamycin resistance-free plate medium to obtain a strain c without the auxiliary gene knockout plasmid; and
(4) transferring a prokaryotic expression vector with the Mtb ATP synthase gene cluster into the strain c to obtain the Mtb ATP synthase-expressing recombinant bacterium.

Preferably, the Mtb ATP synthase gene cluster in step (1) is inserted into a sodC gene locus of the Msm competent cell using streptomycin as a selection marker.

Preferably, the Mtb ATP synthase gene cluster in step (1) has a nucleotide sequence shown in SEQ ID NO: 1.

Preferably, the knocking out in step (2) is conducted using hygromycin as a selection marker.

Preferably, the Msm ATP synthase genome in step (2) has a nucleotide sequence shown in SEQ ID NO: 2.

The present disclosure further provides an Mtb ATP synthase-expressing recombinant bacterium constructed by the construction method.

The present disclosure further provides a method for expressing an Mtb ATP synthase using the Mtb ATP synthase-expressing recombinant bacterium, including: culturing the Mtb ATP synthase-expressing recombinant bacterium to allow induced expression of the Mtb ATP synthase with acetamide.

Preferably, the culturing is conducted at 37° C. with shaking.

Preferably, an obtained cultured bacterial solution is cooled to 16° C. to allow the induced expression with the acetamide.

Preferably, the method further includes the following steps after the induced expression is conducted with the acetamide: collecting a bacterial cell to allow cell disruption, and purifying an obtained Msm cell membrane total protein solution.

Beneficial effects: the present disclosure provides a construction method of an Mtb ATP synthase-expressing recombinant bacterium, including the following steps: transferring an Mtb ATP synthase gene cluster with an affinity purification tag into a Msm competent cell to obtain a strain a; knocking out a Msm ATP synthase genome in the strain a to obtain a strain b; subjecting the strain b to repeated subculture on a kanamycin resistance-free plate medium to obtain a strain c without the auxiliary gene knockout plasmid; and transferring a prokaryotic expression vector with the Mtb ATP synthase gene cluster into the strain c to obtain the Mtb ATP synthase-expressing recombinant bacterium. In the present disclosure, the construction method of the recombinant bacterium solves the problem of difficulty in knocking out background genes of essential genes for heterologous expression, and solves the technical bottleneck that plagues the current scientific research community. The construction method also avoids the problem of formation of heterologous expression hybrid complexes in multi-subunit protein complexes, and provides a solution to the heterozygous expression of essential genes to avoid hybrid.

In the present disclosure, the expression and purification of Mtb ATP synthase are completed using the constructed recombinant bacterium, with an expression mechanism shown in FIG. 1. The exogenous expression of Mtb proteins in Msm avoids the biological hazards of Mtb, and is simple to operate and low in cost with high safety.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 shows a schematic diagram of heterologous expression of the Mtb ATP synthase;

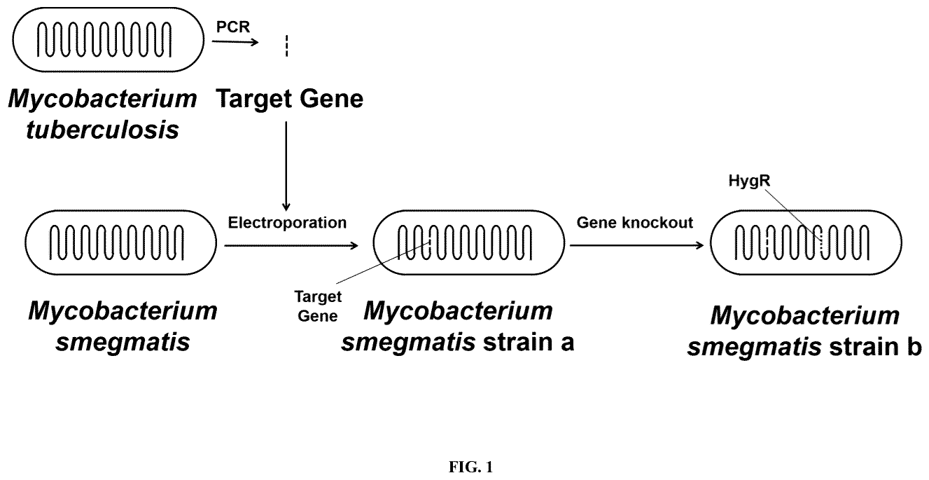

FIG. 2 shows a map of a target plasmid A;

FIG. 3 shows a map of a target plasmid B;

FIG. 4 shows a map of a target plasmid C;

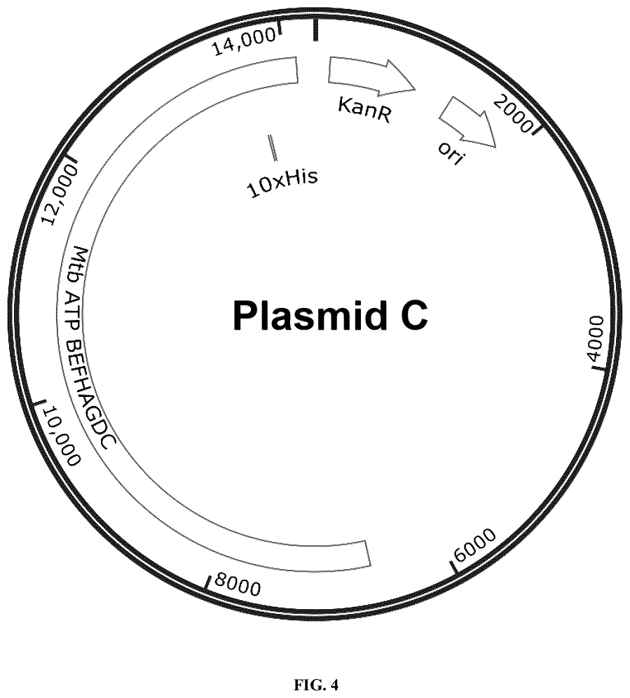

FIG. 5 shows results of gel exclusion chromatography, where an abscissa represents an elution volume of chromatographic column, in mL; and an ordinate represents ultraviolet absorption at 280 nm, in mAU;

FIG. 6 shows results of blue native-polyacrylamide gel electrophoresis (BN-PAGE); and

FIG. 7 shows results of negative-staining transmission electron microscopy (TEM).

### DETAILED DESCRIPTION OF THE EMBODIMENTS

The present disclosure provides a construction method of a Mtb ATP synthase-expressing recombinant bacterium, including the following steps: (1) transferring an Mtb ATP synthase gene cluster with an affinity purification tag into a Msm competent cell to obtain a strain a; where the Msm competent cell carries an auxiliary gene knockout plasmid;
(2) knocking out a Msm ATP synthase genome in the strain a to obtain a strain b;
(3) subjecting the strain b to repeated subculture on a kanamycin resistance-free plate medium to obtain a strain c without the auxiliary gene knockout plasmid; and
(4) transferring a prokaryotic expression vector with the Mtb ATP synthase gene cluster into the strain c to obtain the Mtb ATP synthase-expressing recombinant bacterium.

In the present disclosure, an Mtb ATP synthase gene cluster with an affinity purification tag is transferred into a Msm competent cell to obtain a strain a; where the Msm competent cell carries an auxiliary gene knockout plasmid. The Msm competent cell carries the auxiliary gene knockout plasmid; the auxiliary gene knockout plasmid can assist subsequent gene knockout and facilitate the development of gene knockout. There is no special limitation on a specific type of the auxiliary gene knockout plasmid. In the examples, commercially available pJV53 is used as an example. When constructing Msm containing pJV53, the pJV53 plasmid is preferably electroporated into a Msm mc²155 competent cell treated with an ice bath.

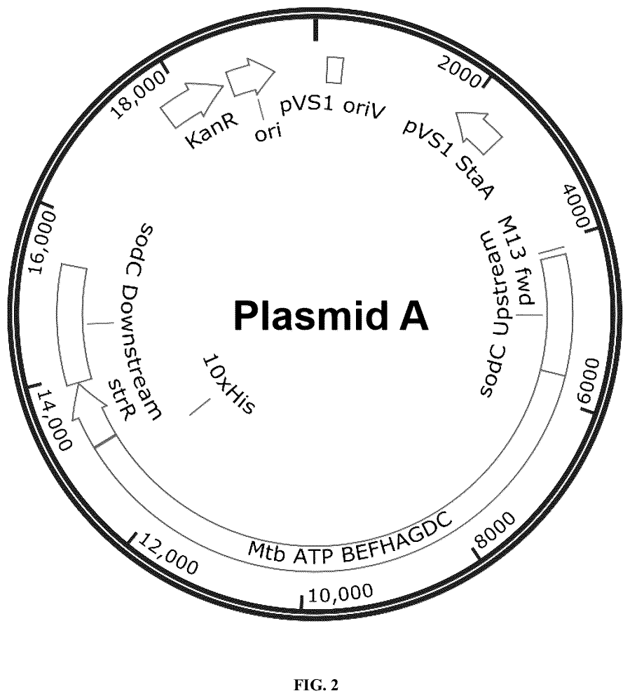

In the present disclosure, there is no special limitation on a method of transfer, which preferably includes homologous recombination, and a transferred gene is the Mtb ATP synthase gene cluster with an affinity purification tag. There is no special limitation on a type of the affinity purification tag, which preferably includes a His tag and a Flag tag. In the examples, a 10×His tag is used as an example for explanation, but cannot be regarded as the entire protection scope of the present disclosure. Preferably, the Mtb ATP synthase gene cluster with an affinity purification tag is inserted into a sod (′ gene locus of the Msm competent cell with streptomycin as a selection marker. The Mtb ATP synthase gene cluster has a nucleotide sequence shown in SEQ ID NO: 1. Specifically, upstream and downstream homologous arms of an sod (′ gene of Msm, the Mtb ATP synthase gene cluster, and a streptomycin gene are transferred into a suicide plasmid pH; a resulting constructed plasmid is transformed into an E. coli DH-5a strain, and a target plasmid A is obtained after sequencing; the target plasmid A is transformed into the Msm competent cell containing the pJV53 plasmid, and a resulting successfully transformed strain is called a strain a. The suicide plasmid pH has a nucleotide sequence preferably shown in SEQ ID NO: 3, and a plasmid map thereof after successful construction is preferably shown in FIG. 2.

In the present disclosure, a Msm ATP synthase genome in the strain a is knocked out to obtain a strain b after the strain a is constructed into the competent cell. The knockout Msm ATP synthase genome has a nucleotide sequence preferably shown in SEQ ID NO: 2, and hygromycin is used as a selection marker in the knocking out. Preferably, the Msm ATP synthase genome in the strain a is knocked out by homologous recombination to obtain the strain b. Specifically, a target plasmid B is constructed, and the target plasmid B is transferred into a competent cell of the strain a to complete the knockout of the Msm ATP synthase genome. A construction process of the target plasmid B preferably includes: transferring upstream and downstream homologous arms of a Msm ATP synthase gene cluster and a hygromycin gene into the suicide plasmid pH; transforming a resulting constructed plasmid into an E. coli DH-5α strain, and obtaining the target plasmid B by sequencing. The target plasmid B is transferred into the competent cell of the strain a to obtain the strain b.

In the present disclosure, the strain b is subjected to repeated subculture on a kanamycin resistance-free plate medium to obtain a strain c without the auxiliary gene knockout plasmid. The repeated subculture is preferably conducted on a kanamycin resistance-free plate. Specifically, the strain b is subjected to repeated subculture on a solid medium containing carbenicillin sodium, streptomycin, and hygromycin until the strain b no longer contains kanamycin resistance, such that the strain b discards the auxiliary gene knockout plasmid to obtain the strain c.

In the present disclosure, a prokaryotic expression vector with the Mtb ATP synthase gene cluster is transferred into the strain c to obtain the Mtb ATP synthase-expressing recombinant bacterium after the strain c is constructed into the competent cell. Preferably, the Mtb ATP synthase gene cluster with an affinity purification tag (Mtb ATP synthase gene cluster for short) is inserted into a base vector of the prokaryotic expression vector. There is no special limitation on the base vector of the prokaryotic expression vector. In the examples, pMV261 is used as an example for explanation, but cannot be considered as the entire protection scope of the present disclosure. For example, in an example, the Mtb ATP synthase gene cluster is inserted into the pMV261 plasmid, and a 10×HIS tag is inserted at a C-terminus of an ATP synthase β subunit; a plasmid is constructed using PCR and homologous recombination, and the plasmid is transformed into an E. coli DH-5α strain, and a target plasmid C is obtained after sequencing; the target plasmid C is transformed into the strain c to obtain the strain d, which is the recombinant bacterium of the present disclosure.

The present disclosure further provides an Mtb ATP synthase-expressing recombinant bacterium constructed by the construction method.

In the present disclosure, the Mtb ATP synthase gene cluster is transferred into Msm using the construction method, and then an ATP synthase gene of the Msm is knocked out. The Msm already contains the Mtb ATP synthase gene cluster with a same function at the time of knockout, thus solving the difficulty in knocking out background genes of essential genes of heterologous expression. Since the background Msm ATP synthase gene cluster is completely deleted, the strain only contains the genes of Mtb, thereby avoiding the formation of heterologous expression hybrid complexes in multi-subunit protein complexes. The overexpression plasmid pMV261 containing the Mtb ATP synthase gene cluster is transferred to further increase a protein expression level to obtain more proteins.

The present disclosure further provides a method for expressing an Mtb ATP synthase using the Mtb ATP synthase-expressing recombinant bacterium, including: culturing the Mtb ATP synthase-expressing recombinant bacterium to allow induced expression of the Mtb ATP synthase with acetamide.

In the present disclosure, the recombinant bacterium is preferably subjected to shaking culture at 37° C. under 220 rpm. In the examples, the recombinant bacterium is cultured under the above conditions for 36 h, cooled to 16° C., and then subjected to induce expression with acetamide.

In the present disclosure, after the induced expression is completed, preferably a bacterial cell is collected to obtain a Msm cell membrane total protein solution through cell disruption, centrifugation, and membrane dissolution; and the Mtb ATP synthase is isolated and purified using nickel ion metal chelate affinity chromatography and gel exclusion chromatography.

In the present disclosure, the bacterial cell is preferably mixed with a buffer A and then lysed, a supernatant is extracted after first centrifugation at 14,000 rpm for 15 min, and a precipitate is collected after second centrifugation at 36,900 rpm for 2 h, so as to obtain a cell membrane. The cell membrane is mixed with a buffer B and ground evenly, added with a detergent, shaken in an ice bath for 2 h, and centrifuged at 18,000 rpm for 40 min, and a supernatant obtained is the cell membrane protein solution after membrane dissolution. The buffer A for the cell disruption preferably includes: 20 mM 3-morpholinopropane sulfonic acid, 100 mM sodium chloride, 5 mM magnesium chloride, 5 mM 6-aminocaproic acid, 5 mM benzamidine, and 1 mM PMSF, pH=7.4. The detergent preferably includes LMNG, and the buffer B for the membrane dissolution preferably includes: 20 mM 3-morpholinopropane sulfonic acid, 100 mM sodium chloride, 5 mM magnesium chloride, 5 mM 6-aminocaproic acid, 5 mM benzamidine, 1 mM PMSF, and 0.004% W/V LMNG, pH=7.4.

In the present disclosure, the nickel ion metal chelate affinity chromatography preferably uses a buffer C to wash impurities; the buffer C preferably includes: 20 mM 3-morpholinopropane sulfonic acid, 100 mM sodium chloride, 50 mM imidazole, 5 mM magnesium chloride, 5 mM 6-aminocaproic acid, 5 mM benzamidine, 1 mM PMSF, and 0.004% W/V LMNG, pH=7.4. The nickel ion metal chelate affinity chromatography preferably uses a buffer D to conduct elution; the buffer D preferably includes: 20 mM 3-morpholinopropane sulfonic acid, 100 mM sodium chloride, 500 mM imidazole, 5 mM magnesium chloride, 5 mM 6-aminocaproic acid, 5 mM benzamidine, 1 mM PMSF, and 0.004% W/V LMNG, pH=7.4.

In the present disclosure, the eluate is preferably concentrated using a concentration tube with a molecular weight cut-off of 100 KDa, and a concentrate is subjected to gel exclusion chromatography. The buffer E used in the gel exclusion chromatography preferably includes: 20 mM 3-morpholinopropane sulfonic acid, 100 mM sodium chloride, 5 mM magnesium chloride, and 0.004% W/V LMNG, pH=7.4.

In order to further illustrate the present disclosure, the Mtb ATP synthase-expressing recombinant bacterium, and the construction method and the expression method thereof provided by the present disclosure will be described in detail below in conjunction with accompanying drawings and examples, but they should not be construed as limiting the protection scope of the present disclosure.

In the present disclosure, the test materials and methods used in the examples are all test materials and methods known in the art unless otherwise specified. The specific composition of the media and buffers used in the examples are as follows:

A culture includes: 10 g tryptone, 10 g sodium chloride, 5 g yeast powder, and 1 g Tween 80 are diluted with water to 1 L, sterilized with high-pressure steam for 30 min, and allowed to cool for later use.

A solid culture includes: 10 g tryptone, 10 g sodium chloride, 5 g yeast powder, and 15 g agar are diluted with water to 1 L, sterilized with high-pressure steam for 30 min, cooled to 50° C., added with antibiotics, and poured into a petri dish for later use. Each dish has about 20 mL of a solid medium liquid.

A buffer A includes: 20 mM 3-morpholinopropane sulfonic acid, 100 mM sodium chloride, 5 mM magnesium chloride, 5 mM 6-aminocaproic acid, 5 mM benzamidine, and 1 mM PMSF; pH=7.4;
a buffer B includes: 20 mM 3-morpholinopropane sulfonic acid, 100 mM sodium chloride, 5 mM magnesium chloride, 5 mM 6-aminocaproic acid, 5 mM benzamidine, 1 mM PMSF, and 0.004% W/V LMNG; pH=7.4;
a buffer C includes: 20 mM 3-morpholinopropane sulfonic acid, 100 mM sodium chloride, 50 mM imidazole, 5 mM magnesium chloride, 5 mM 6-aminocaproic acid, 5 mM benzamidine, 1 mM PMSF, and 0.004% W/V LMNG; pH=7.4;
a buffer D includes: 20 mM 3-morpholinopropane sulfonic acid, 100 mM sodium chloride, 500 mM imidazole, 5 mM magnesium chloride, 5 mM 6-aminocaproic acid, 5 mM benzamidine, 1 mM PMSF, and 0.004% W/V LMNG; pH=7.4; and
a buffer E includes: 20 mM 3-morpholinopropane sulfonic acid, 100 mM sodium chloride, 5 mM magnesium chloride, and 0.004% W/V LMNG; pH=7.4.

### Example 1

(I) Construction of Msm Competent Cells Containing Plasmid pJV53
(1) Construction of Msm Glycerol Stock Containing Plasmid pJV53

A tube of Msm competent cells was thawed on ice, added with 1 μg of plasmid pJV53 on an ultra-clean bench, incubated on ice for 30 min, and electroporated at 2,500 V for 5.7 ms to allow the plasmid to enter the cells. The cells were placed on ice for 5 min, added into 1 mL of sterilized medium, and then revived at 180 rpm and 37° C. for 4 h. The cells were centrifuged at 4,000 rpm for 10 min, 1 mL of a supernatant was discarded, and the remaining supernatant was mixed with the pellet, and then spread evenly with a spreading rod on a solid medium containing 50 μg/mL kanamycin and 40 μg/mL carbenicillin sodium. The cells were incubated at 37° C. in an incubator for three days.

(2) 5 μL of 50 mg/mL kanamycin and 5 μL of 40 mg/mL carbenicillin sodium were mixed well in 5 mL of medium, the Msm containing plasmid pJV53 was selected from the cultured plate, and subjected to shaking culture at 37° C. and 220 rpm for 48 h. 200 μL of 50 mg/mL kanamycin and 200 μL of 40 mg/mL carbenicillin sodium were mixed well in 200 mL medium, and 5 mL of cultured Msm containing plasmid pJV53 was added to allow shaking culture at 37° C. and 220 rpm for about 5 h to 8 h until a bacterial concentration reached an OD₆₀₀ value of about 0.5. 1 mL of 40% (w/v) acetamide solution was added to a resulting bacterial solution to allow shaking culture at 37° C. and 220 rpm for about 4 h. The cultured bacterial cells were bathed in ice water for 2.5 h.

The bacterial solution was centrifuged under sterile conditions at 3,200 rpm and 4° C. for 10 min, a supernatant was discarded, 30 mL of 10% glycerol in ice bath after sterilization was added, and the bacterial solution was gently suspended and centrifuged at 3,200 rpm and 4° C. for 10 min, a supernatant was discarded; and the above glycerol washing operations were repeated three times.

8 mL of sterilized 10% glycerol in ice bath was added to the washed bacterial cells, gently suspended, mixed well, and 180 μL of the competent cells were distributed into each tube.

The competent cells were quickly frozen in liquid nitrogen and stored at −80° C.

(II) Construction of Msm Competent Cells Transferred with Mtb ATP Synthase

(1) The upstream and downstream homologous arms of the sod (′ gene of Msm, the Mtb ATP synthase gene cluster, and the streptomycin gene were transferred into the suicide plasmid pH, and the plasmid was constructed using PCR and homologous recombination. The original PH plasmid had a sequence shown in SEQ ID NO: 3. The 1,500 bp upstream homologous arm was amplified using Msm as a template and primers sodC UP-F/sodC UP-R; the 1,500 bp downstream homologous arm was amplified using Msm as a template and primers sodC DOWN-F/sodC DOWN-R; the streptomycin gene of approximately 800 bp was amplified using the PH plasmid as a template and primers STR-F/STR-R; and the approximately 6,800 bp linearized PH vector was amplified using the PH plasmid as a template and primers PH-F/PH-R. The amplified and purified fragments by PCR were ligated using a commercial homologous recombinase according to the instructions. The plasmid was transformed into an E. coli DH-5α strain, and the target plasmid A shown in FIG. 2 was obtained by sequencing. The primers were shown in Table 1, and an amplification program included: initial denaturation at 95° C. for 10 min; denaturation at 95° C. for 15 s, annealing at 65° C. for 15 s, and extension at 72° C. for 30 s/kb, 35 cycles; supplementary extension at 72° C. for 10 min; and storage at 4° C.

A tube of Msm competent cells containing pJV53 plasmid was thawed on ice, added with 1 μg of target plasmid A on an ultra-clean bench, incubated on ice for 30 min, and electroporated at 2,500 V for 5.7 ms to allow the plasmid to enter the cells. The cells were placed on ice for 5 min, added into 1 mL of sterilized medium, and revived at 180 rpm and 37° C. for 4 h. The cells were centrifuged at 4,000 rpm for 10 min, 1 mL of a supernatant was discarded, and the remaining supernatant was mixed with the pellet, and then spread evenly with a spreading rod on a solid medium containing 50 μg/mL kanamycin, 40 μg/mL carbenicillin sodium, and 25 μg/mL streptomycin. The cells were incubated at 37° C. in an incubator for three days.

5 μL of 50 mg/mL kanamycin, 5 μL of 40 mg/mL carbenicillin sodium, and 2.5 μL of 50 mg/mL streptomycin were mixed well in 5 mL of medium, a single colony of the Msm was selected from the cultured plate, and subjected to shaking culture at 37° C. and 220 rpm for 48 h.

A fragment of about 9,000 bp was amplified by PCR using primers A-F/A-R in Table 1 to detect whether the target plasmid A was integrated into the genome. A PCR product was sequenced, and the completely correct strain was preserved as the strain a.

TABLE 1
Primers for constructing plasmid A and sequencing strain a

| | | Number | SEQ |
|---|---|---|---|
| Primer | | of | ID |
| name | Primer sequence (5′ to 3′) | bases | NO: |
| PH-F | GATATCCTCAATTCCGTCGGTCGAT | 25 | 5 |
| PH-R | TCTAGAAGTCCTCGTGTACTGTGTAAG | 27 | 6 |
| sodC UP-F | AGTACACGAGGACTTCTAGAAgccgttgcgtttgacggcc | 40 | 7 |
| sodC UP-R | gccaggatggtctcagtcatggcccaaagcctagccggtgac | 42 | 8 |
| sodC | ATCGATATCcagccggatcgacttcgccgggtcaccccggc | 41 | 9 |
| DOWN-F | | | |
| sodC | CCGACGGAATTGAGGATATCccagggtgacccacagcaac | 40 | 10 |
| DOWN-R | | | |
| STR-F | gcgcgccgtcggcgcgatcgactaagaattctagaggtccgctttgaatc | 50 | 11 |
| STR--R | cggcgaagtcgatccggctgGATATCGATCACTAGTTCAA | 40 | 12 |
| TBATP-F | gtcaccggctaggctttgggccatgactgagaccatcctggc | 42 | 13 |
| TBATP-R | aaagcggacctctagaattcttagtcgatcgcgccgacggcgcgcaatct | 50 | 14 |
| A-F | aaccgtctgcgtgaggcggagtgggacgtgaccga | 35 | 15 |
| A-R | cggccgaccacttgccgaacggatgggttcccgca | 35 | 16 |

(2) Preparing Strain a into Competent Cells

The preserved strain a was streaked on a solid medium plate containing 50 μg/mL kanamycin, 40 μg/mL carbenicillin sodium, and 25 μg/mL streptomycin, and incubated in a constant-temperature incubator at 37° C. for three days.

5 μL of 50 mg/mL kanamycin, 5 μL of 40 mg/mL carbenicillin sodium, and 2.5 μL of 50 mg/mL streptomycin were mixed well in 5 mL of medium, a single colony was selected from the cultured plate, and subjected to shaking culture at 37° C. and 220 rpm for 48 h. 200 μL of 50 mg/mL kanamycin, 200 μL of 40 mg/mL carbenicillin sodium, and 100 μL of 50 mg/mL streptomycin were mixed well in 200 mL medium, and 5 mL of cultured strain a was added to allow shaking culture at 37° C. and 220 rpm for about 5 h to 8 h until a bacterial concentration reached an OD₆₀₀ value of about 0.5. 1 mL of 40% (w/v) acetamide solution was added to a resulting bacterial solution to allow shaking culture at 37° C. and 220 rpm for about 4 h. The cultured bacterial cells were bathed in ice water for 2.5 h.

The bacterial solution was centrifuged under sterile conditions at 3,200 rpm and 4° C. for 10 min, a supernatant was discarded, 30 mL of 10% glycerol in ice bath after sterilization was added, and the bacterial solution was gently suspended and centrifuged at 3,200 rpm and 4° C. for 10 min, a supernatant was discarded; and the above glycerol washing operations were repeated three times.

8 mL of sterilized 10% glycerol in ice bath was added to the washed bacterial cells, gently suspended, mixed well, and 180 μL of the competent cells were distributed into each tube.

The competent cells were quickly frozen in liquid nitrogen and stored at −80° C.

(III) Construction of Msm Competent Cells with Knockout of Msm ATP Synthase

(1) The upstream and downstream homologous arms of the Msm ATP synthase gene cluster and the hygromycin gene were transferred into the suicide plasmid pH. The 1,500 bp upstream homologous arm was amplified using primers MsmATP UP-F/MsmATP UP-R; the 1,500 bp downstream homologous arm was amplified using primers MsmATP DOWN-F/Msm ATP DOWN-R; the hygromycin gene of approximately 1,000 bp was amplified using HYG plasmid (with a sequence shown in SEQ ID NO: 4) as a template and primers HYG-F/HYG-R; and the approximately 6,800 bp linearized PH vector was amplified using the PH plasmid as a template and primers PH-F/PH-R. The amplified and purified fragments by PCR were ligated using a commercial homologous recombinase according to the instructions. The plasmid was transformed into an E. coli DH-5α strain, and the target plasmid A shown in FIG. 2 was obtained by sequencing. The primers used were shown in Table 2, and the amplification procedure was the same as above.

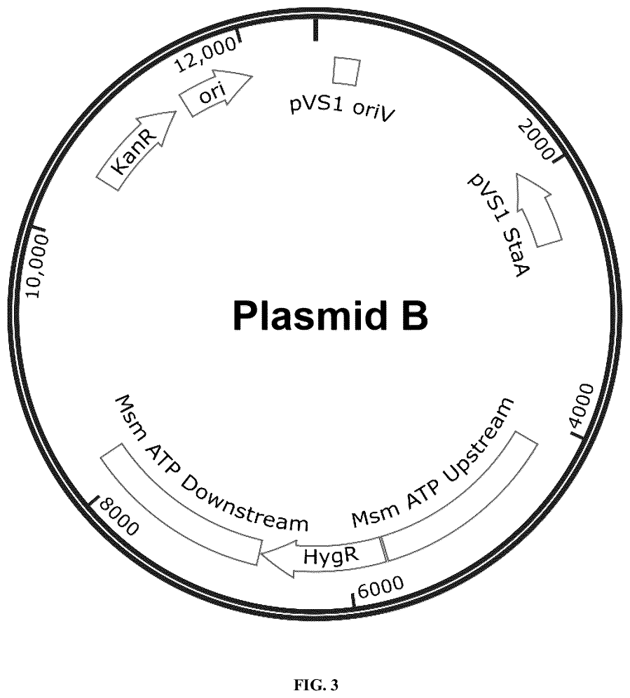

A plasmid was constructed using PCR and homologous recombination, and the plasmid was transformed into an E. coli DH-5α strain, and the target plasmid B shown in FIG. 3 was obtained by sequencing.

A tube of strain a competent cells was thawed on ice, added with 1 μg of target plasmid B on an ultra-clean bench, incubated on ice for 30 min, and electroporated at 2,500 V for 5.7 ms to allow the plasmid to enter the cells. The cells were placed on ice for 5 min, added into 1 mL of sterilized medium, and revived at 180 rpm and 37° C. for 4 h. The cells were centrifuged at 4,000 rpm for 10 min, 1 mL of a supernatant was discarded, and the remaining supernatant was mixed with the pellet, and then spread evenly with a spreading rod on a solid medium containing 50 μg/mL kanamycin, 40 μg/mL carbenicillin sodium, 25 μg/mL streptomycin, and 50 μg/mL hygromycin. The cells were incubated at 37° C. in an incubator for three days.

5 μL of 50 mg/mL kanamycin, 5 μL of 40 mg/mL carbenicillin sodium, 2.5 μL of 50 mg/mL streptomycin, and 2.5 μL of 100 mg/mL hygromycin were mixed well in 5 mL of medium, a single colony of the Msm was selected from the cultured plate, and subjected to shaking culture at 37° C. and 220 rpm for 48 h.

A fragment of about 4,500 bp was amplified by PCR using primers B-F/B-R in Table 2 to detect whether the target plasmid B was integrated into the genome. A PCR product was sequenced, and the completely correct strain was preserved as the strain b.

TABLE 2
Primers for constructing plasmid B and sequencing strain b

| | | Number | SEQ |
|---|---|---|---|
| Primer | | of | ID |
| name | Primer sequence (5′ to 3′) | bases | NO: |
| PH-F | GATATCCTCAATTCCGTCGGTCGATTACAATCACATGACTTGGCT | 45 | 17 |
| PH-R | TCTAGAAGTCCTCGTGTACTGTGTAAGCGCCCACTCCACAT | 41 | 18 |
| HYG-F | GAATTCtagaggtccgctgtgacacaagaat | 31 | 19 |
| HYG-R | GATATCGATCACTAGTtcaggcgccgggggcggtg | 35 | 20 |
| MsmATP UP-F | CACAGTACACGAGGACTTCTAGAggtggcggcggtcttga | 40 | 21 |
| MsmATP UP-R | gtcacagcggacctctaGAATTCggtcgttgtctgagtcatggaggt | 47 | 22 |
| MsmATP | ACTAGTGATCGATATCtcacatcgatgagcgcgtccatgatcggca | 46 | 23 |
| DOWN-F | | | |
| MsmATP | GACGGAATTGAGGATATCgtgcatgtccagcggccgcgac | 40 | 24 |
| DOWN-R | | | |
| B-F | tgcgttgatacatcgcccaggtggcctcgatctcggcgt | 39 | 29 |
| B-R | tgctgcgggtcgacgccggtgaccgtgatgtcacccct | 38 | 30 |

(2) The strain b was subjected to repeated subculture on a solid medium containing 40 μg/mL carbenicillin sodium, 25 μg/mL streptomycin, and 50 μg/mL hygromycin until the strain b no longer contained kanamycin resistance, such that the strain b discarded the pJV53 plasmid to obtain the strain c.

(3) The strain c was prepared into competent cells. The preserved strain c was streaked on a solid medium plate containing 40 μg/mL carbenicillin sodium, 25 μg/mL streptomycin, and 50 μg/mL hygromycin, and incubated in a constant-temperature incubator at 37° C. for three days.

5 μL of 40 mg/mL carbenicillin sodium, 2.5 μL of 50 mg/mL streptomycin, and 2.5 μL of 100 mg/mL hygromycin were mixed well in 5 mL of medium, a single colony was selected from the cultured plate, and subjected to shaking culture at 37° C. and 220 rpm for 48 h. 200 μL of 40 mg/mL carbenicillin sodium, 100 μL of 50 mg/mL streptomycin, and 100 μL of 100 mg/mL hygromycin were mixed well in 200 mL medium, and 5 mL of cultured strain b was added to allow shaking culture at 37° C. and 220 rpm for about 5 h to 8 h until a bacterial concentration reached an OD₆₀₀ value of about 0.5. 1 mL of 40% (w/v) acetamide solution was added to a resulting bacterial solution to allow shaking culture at 37° C. and 220 rpm for about 4 h. The cultured bacterial cells were bathed in ice water for 2.5 h.

The bacterial solution was centrifuged under sterile conditions at 3,200 rpm and 4° C. for 10 min, a supernatant was discarded, 30 mL of 10% glycerol in ice bath after sterilization was added, and the bacterial solution was gently suspended and centrifuged at 3,200 rpm and 4° C. for 10 min, a supernatant was discarded; and the above glycerol washing operations were repeated three times.

8 mL of sterilized 10% glycerol in ice bath was added to the washed bacterial cells, gently suspended, mixed well, and then 180 μL of the competent cells were distributed into each tube.

The competent cells were quickly frozen in liquid nitrogen and stored at −80° C.

(IV) Construction and Transferring of pMV261-Mtb ATP Plasmid

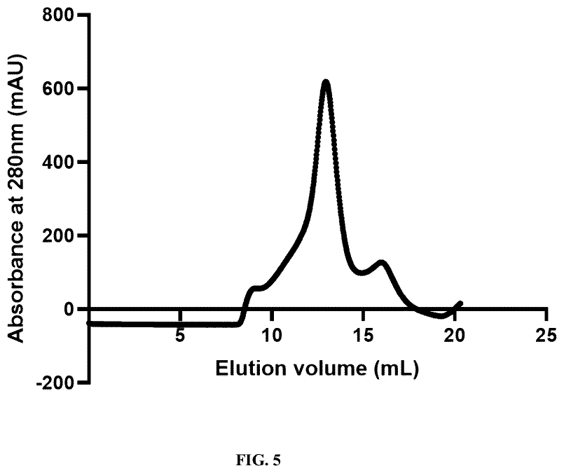

The Mtb ATP synthase gene cluster was inserted into the pMV261 plasmid, and a 10×HIS tag was inserted at a C-terminus of an ATP synthase β subunit; a plasmid was constructed using PCR and homologous recombination with primers shown in Table 3. The Mtb ATP synthase gene cluster of approximately 7,500 bp was amplified using plasmid A as a template and primers 261-TBATP-F/261-TBATP-R; the linearized pMV261 plasmid of approximately 6,800 bp was amplified using the pMV261 plasmid as a template and primers P261F/P261R. The amplified and purified fragments by PCR were ligated using a commercial homologous recombinase according to the instructions. The plasmid was transformed into an E. coli DH-5α strain, and the target plasmid C shown in FIG. 5 was obtained by sequencing.

A tube of strain c competent cells was thawed on ice, added with 1 μg of target plasmid C on an ultra-clean bench, incubated on ice for 30 min, and electroporated at 2,500 V for 5.7 ms to allow the plasmid to enter the cells. The cells were placed on ice for 5 min, added into 1 mL of sterilized medium, and revived at 180 rpm and 37° C. for 4 h. The cells were centrifuged at 4,000 rpm for 10 min, 1 mL of a supernatant was discarded, and the remaining supernatant was mixed with the pellet, and then spread evenly with a spreading rod on a solid medium containing 50 μg/mL kanamycin, 5 μL of 40 μg/mL carbenicillin sodium, 25 μg/mL streptomycin, and 50 μg/mL hygromycin. The cells were incubated at 37° C. in an incubator for three days.

5 μL of 50 mg/mL kanamycin, 5 μL of 40 mg/mL carbenicillin sodium, 2.5 μL of 50 mg/mL streptomycin, and 2.5 μL of 100 mg/mL hygromycin were mixed well in 5 mL of medium, a single colony of the Msm was selected from the cultured plate, and subjected to shaking culture at 37° C. and 220 rpm for 48 h. A final expression strain d was obtained by retaining the glycerol stock.

TABLE 3
Primers for constructing plasmid C

| | | Number | SEQ |
|---|---|---|---|
| Primer | | of | ID |
| name | Primer sequence (5′ to 3′) | bases | NO: |
| 261-TBATP-F | AGTTGGATCCTACTTCCAATCCAATGCTatgactga | 48 | 31 |
| | gaccatcctggc | | |
| 261-TBATP-R | CTTTTATCCCACCCAAATGttagtcgatcgcgccgacggc | 46 | 32 |
| | gcgcaa | | |
| P261F | CATTTGGGTGGGATAA | 16 | 33 |
| p261R | AGCATTGGATTGGAAGTA | 18 | 34 |

### (V) Culture of Expression Strains and Collection of Cell Membrane Proteins

50 μL of 40 mg/mL carbenicillin sodium, 50 μL of 50 mg/mL kanamycin, 25 μL of 50 mg/mL streptomycin, and 25 μL of 100 mg/mL hygromycin were mixed well in 50 mL of medium, 1 mL of a glycerol stock of the strain d was selected from the cultured plate, and subjected to shaking culture at 37° C. and 220 rpm for 24 h. The strain was expanded into 1 L culture medium, 1 mL of 40 mg/mL carbenicillin sodium, 1 mL of 50 mg/mL kanamycin, 500 μL of 50 mg/mL streptomycin, and 500 μL of 100 mg/mL hygromycin were mixed well in each 1 L medium, and 5 mL of cultured strain b was added to allow shaking culture at 37° C. and 220 rpm for about 36 h. 5 mL of 40% (w/v) acetamide solution was added to a resulting bacterial solution to allow shaking culture at 37° C. and 220 rpm for about 4 d. The bacterial solution was centrifuged at 4,000 rpm for 15 min to collect the bacteria, the supernatant was discarded, and the precipitate was retained for later use.

100 mL of the buffer A was added to every 20 g of the precipitate, mixed evenly, and the cells were lysed with a high-pressure homogenizer. The cells were centrifuged at 14,000 rpm for 15 min at 4° C., and the supernatant was collected for later use. The supernatant was centrifuged at 36,900 rpm for 2 h at 4° C., a resulting new supernatant was discarded, and the remaining precipitate was the cell membrane.

The cell membrane was ground in a tissue grinder using 30 mL of the buffer B evenly, 4 mL of 10% (w/v) LMNG was added, and diluted to 40 mL and shaken in an ice bath for 2 h.

A resulting solution was centrifuged at 18,000 rpm for 40 min at 4° C., a resulting supernatant was the cell membrane protein solution after membrane dissolution, and then collected for later use.

### (VI) Purification of Mtb ATP Synthase

50 mL of the buffer B was added to a gravity column filled with nickel ion metal chelate affinity chromatography medium to allow equilibrium, and the liquid was allowed to drain naturally. The cell membrane protein solution naturally flowed through the gravity column four times. The gravity column was washed with 100 mL of the buffer C, and the protein was eluted with 15 mL of the buffer D.

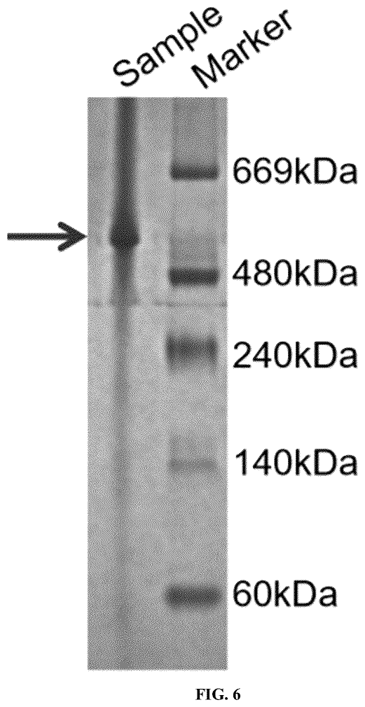

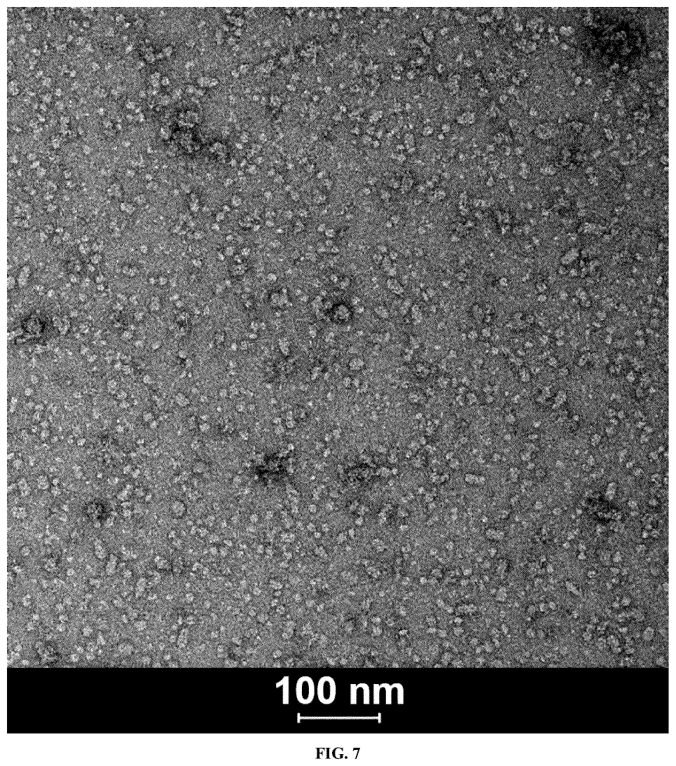

A resulting eluate was concentrated using a concentrator tube with a molecular weight cut-off of 100 KDa. 24 mL of Superose 6 increase 10/300GE gel exclusion chromatography column was equilibrated with 25 mL buffer E; the prepared protein was loaded onto the column, and the Mtb ATP synthase protein was collected. The gel exclusion chromatography results of the prepared pure protein solution were shown in FIG. 5. The gel exclusion chromatography showed a single peak, indicating that the protein was relatively homogeneous; a peak appeared at 13 mL; compared with the Marker, the protein was about 500 kDa in size, which was close to a theoretical molecular weight of 560 kDa. BN-PAGE electrophoresis showed that the complex was uniform, with a purity of over 90%, and the yield was determined to be approximately 0.5 mg (FIG. 6). Electron microscopy results showed that the ATP synthase had a complete structure. FIG. 7 showed data of the ATP synthase collected by negative-staining TEM. The purified protein could bind to bedaquiline, a targeted inhibitor of the ATP synthase, and a protein-small molecule binding constant measured using SPR was 36.48 μM.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A construction method of a Mycobacterium tuberculosis (Mtb) adenosine triphosphate (ATP) synthase-expressing recombinant bacterium, comprising the following steps:
(1) transferring an Mtb ATP synthase gene cluster with an affinity purification tag into a Mycobacterium smegmatis (Msm) competent cell to obtain a strain a; wherein the Msm competent cell carries an auxiliary gene knockout plasmid;
(2) knocking out a Msm ATP synthase genome in the strain a to obtain a strain b;
(3) subjecting the strain b to repeated subculture on a kanamycin resistance-free plate medium to obtain a strain c without the auxiliary gene knockout plasmid; and
(4) transferring a prokaryotic expression vector with the Mtb ATP synthase gene cluster into the strain c to obtain the Mtb ATP synthase-expressing recombinant bacterium.

2. The construction method according to claim 1, wherein the Mtb ATP synthase gene cluster in step (1) is inserted into a sodC gene locus of the Msm competent cell using streptomycin as a selection marker.

3. The construction method according to claim 1, wherein the Mtb ATP synthase gene cluster in step (1) has a nucleotide sequence shown in SEQ ID NO: 1.

4. The construction method according to claim 2, wherein the Mtb ATP synthase gene cluster in step (1) has a nucleotide sequence shown in SEQ ID NO: 1.

5. The construction method according to claim 1, wherein the knocking out in step (2) is conducted using hygromycin as a selection marker.

6. The construction method according to claim 1, wherein the Msm ATP synthase genome in step (2) has a nucleotide sequence shown in SEQ ID NO: 2.

7. The construction method according to claim 5, wherein the Msm ATP synthase genome in step (2) has a nucleotide sequence shown in SEQ ID NO: 2.

8. An Mtb ATP synthase-expressing recombinant bacterium constructed by the construction method according to claim 1.

9. An Mtb ATP synthase-expressing recombinant bacterium constructed by the construction method according to claim 2.

10. An Mtb ATP synthase-expressing recombinant bacterium constructed by the construction method according to claim 3.

11. An Mtb ATP synthase-expressing recombinant bacterium constructed by the construction method according to claim 4.

12. An Mtb ATP synthase-expressing recombinant bacterium constructed by the construction method according to claim 5.

13. An Mtb ATP synthase-expressing recombinant bacterium constructed by the construction method according to claim 6.

14. An Mtb ATP synthase-expressing recombinant bacterium constructed by the construction method according to claim 7.

15. A method for expressing an Mtb ATP synthase using the Mtb ATP synthase-expressing recombinant bacterium according to claim 8, comprising: culturing the Mtb ATP synthase-expressing recombinant bacterium to allow induced expression of the Mtb ATP synthase with acetamide.

16. A method for expressing an Mtb ATP synthase using the Mtb ATP synthase-expressing recombinant bacterium according to claim 9, comprising: culturing the Mtb ATP synthase-expressing recombinant bacterium to allow induced expression of the Mtb ATP synthase with acetamide.

17. A method for expressing an Mtb ATP synthase using the Mtb ATP synthase-expressing recombinant bacterium according to claim 10, comprising: culturing the Mtb ATP synthase-expressing recombinant bacterium to allow induced expression of the Mtb ATP synthase with acetamide.

18. The method according to claim 15, wherein the culturing is conducted at 37° C. with shaking.

19. The method according to claim 15, wherein an obtained cultured bacterial solution is cooled to 16° C. to allow the induced expression with the acetamide.

20. The method according to claim 15, further comprising the following steps after the induced expression is conducted with the acetamide: collecting a bacterial cell to allow cell disruption, and purifying an obtained Msm cell membrane total protein solution.
